(19) United States
(12) Patent Application Publication
(10) Pub. No.: US 2005/0037447 A1
(43) Pub. Date: Feb. 17, 2005

(54) Protein markers for human benign prostatic hyperplasia (BPH)

(76) Inventors: Yong-Chuan Wong, Hong Kong (CN); Ke-Xin Xu, Beijing (CN); Xiang-Hong Wang, Hong Kong (CN)

Correspondence Address: Robert D. Katz, Esq;COOPER & DUNHAM LLP, 1185 Avenue of the Americas, New York, NY, 10036, US

(21) Appl. No.: 10/881,625

(22) Filed: Jun. 30, 2004

Related U.S. Application Data
(60) Provisional application No. 60/485,653, filed on Jul. 8, 2003.
(60) Provisional application No. 60/498,623, filed on Aug. 27, 2003.

(51) Int. Cl. G01N 33/53; G01N 33/567; G01N 33/574

(52) U.S. Cl. 435/7.23

(57) ABSTRACT

This invention provides a method for determining whether a subject is afflicted with Benign Prostate Hyperplasia (BPH) comprising obtaining a sample of prostatic fluid from the subject and analyzing the sample of prostatic fluid to detect the presence of BPH protein markers to determine whether the subject is afflicted with BPH. This invention further provides a method for determining whether a subject is afflicted with Benign Prostate Hyperplasia (BPH) comprising obtaining a serum sample from the subject and analyzing the serum sample to detect the presence of BPH protein markers to determine whether the subject is afflicted with BPH. Finally, this invention provides a diagnostic kits for detecting Benign Prostate Hyperplasia (BPH) markers comprising BPH protein markers and instructions for use.

This application claims priority of U.S. Provisional Application No. 60/485,653, filed Jul. 8, 2003 and U.S. Provisional Application No. 60/498,623, filed Aug. 27, 2003, the contents of which are hereby incorporated by reference into this application.

Throughout this application, various publications may be referenced by author name and date in parentheses. Full citations for these publications may be found at the end of the specification immediately preceding the claims. The disclosures of these publications in their entireties are hereby incorporated by reference into this application in order to more fully describe the state of the art as known to those skilled therein as of the date of the invention described and claimed herein.

### FIELD OF THE INVENTION

This invention relates to novel protein markers for detecting benign prostatic hyperplasia (BPH) and to methods for detecting BPH.

### BACKGROUND OF THE INVENTION

Benign Prostatic Hyperplasia (BPH) is a common derangement of the prostate gland related to increasing age. A very high percentage of males over the age of fifty (68%) have histological evidence of benign prostatic hyperplasia and by age seventy, forty three percent (43%) of males have clinically palpable prostatic enlargement. This disease is characterized by gradual increase in both glandular and fibromuscular tissue in the peri-urethral and transitional zones of the prostate resulting in symptoms of bladder outflow obstruction. In the US alone, between 350,000 and 400,000 surgeries are performed each year for BPH making it the most common procedure in the US at a total cost of $4.5 billion. The incidence of BPH is far higher than prostate cancer but the etiology is little known. However, it is generally believed that a subtle change in balance between testosterone and estrogen in men with advancing age may trigger the growth of the prostate gland resulting in BPH. Prostate enlargement from benign (or malignant) disease generally results in a reduced compliance of the prostatic urethra and increasing urine outflow obstruction. Secondary changes often occur in the bladder as a result. These consist of hypertrophy of bladder wall and formation of trabeculae and diverticula. The secondary changes in detrusor muscle may be largely responsible for the most troublesome symptoms associated with prostatic obstruction: nocturia, urination frequency and urgency. The underlying mechanism remains controversial, but is probably in part associated with the development of detrusor instability in the obstructed bladder.

BPH resulting in bladder outflow obstruction is traditionally treated by transurethral resection of the prostate (TURP). Although TURP is now considered the gold standard for the surgical management of BPH, there is high incidence of complications. For example, 70% of resected cases suffer from different degrees of retrograde ejaculation, and 1-2% result in urinary incontinence. Alternatively, BPH can be treated by drugs such as some α-blockers or by finasteride (Proscar) to block the activity of testosterone on prostate. The latter drug works by inhibiting the activity of 5-α reductase to prevent the conversion of testosterone to DHT, the active form in the prostate gland. By doing so, it prevents the prostate from being stimulated by androgen and prevents it from further growth, thus preventing development of clinical BPH. However, there is no reliable marker to detect BPH before it grows to detectable size or develops symptoms that requires surgical intervention. By then, in most cases, the damage to the bladder detrusor has occurred and a significant number may not improve even after TURP. The existing well known markers, prostatic acid phosphates (PAP) and prostatic specific antigen (PSA), though useful in detection of prostate cancer, are not very useful in early detection of BPH. PSA is currently the best and most useful marker for prostate cancer. However, it cannot accurately differentiate BPH from prostate cancer because of a great deal of overlapping in PSA levels between BPH and prostate cancer, especially for borderline cases (with PSA levels ranging from 4-10 ng/ml). Attempts have been made to improve the accuracy of diagnosis in the borderline zone by measuring the ratio between free PSA (PSA not bound to protease inhibitor) and total PSA (free PSA plus PSA bound to α-1 antichymotrypsin [ACT]), or a combination of PSA and hK2. However, a significant number of patients are still wrongly classed. Some attempts have been made recently by several laboratories worldwide focusing on specific markers for BPH. Mikolajczyk and his colleagues have reported an elevation of a modified form of free PSA in transitional zone prostatic tissue from BPH patients that they referred to as BPSA (BPH-associated PSA). They have further reported the occurrence of BPSA in seminal plasma. The potential of BPSA as a marker for BPH is being actively explored. The present study of prostate carcinogenesis establishes that prostate cancer can be readily induced by implantation of a combination of testosterone (T) and estradiol benzoate (E₂) subcutaneously. This is associated with concurrent changes in profile of secretory 10 proteins, altered expression of a number of peptide growth factors, derangement in stromal smooth muscle and differential gene expression, including over expression of Id-1 gene during the development and progression of prostate cancer in both the animal model and human prostate cancer. The function of Id-1 gene has been extensively studied by transfection of this gene into a well-known human prostate cancer cell line, LNCaP, and found to stimulate cancer cell proliferation in serum free medium through inactivation of p16^{INK4a}/pRB, which involves the activation of MAPK and NF-κB pathways. Using the hormone-induced Noble rat model of prostate carcinogenesis, it was found that dysplasia is first detected in prostate gland 2-3 months after hormone capsules implantation. The development of dysplasia/carcinoma coincides with the appearance of a new secretory protein, a kallikrein-like protein which is homologous to human kallikrein 2 (hK2) (21) and a member of the prostate specific antigen (PSA) gene family. Despite the usefulness of PSA (hK3) in diagnosis of prostate cancer, it is not tumor specific. A worldwide extensive search is on for a better and more specific tumor marker for the prostate gland in which hK2 is one of the contenders, as it appears to be more tumor specific with its level more directly and linearly related to prostate cancer. The present study demonstrates that similar changes in secretory protein profile occur in human BPH, where specific proteins may be secreted that can be detected in prostatic secretion and/or serum.

### SUMMARY OF THE INVENTION

This invention provides a method for determining whether a subject is afflicted with Benign Prostate Hyperplasia (BPH) comprising obtaining a sample of prostatic fluid from the subject and analyzing the sample of prostatic fluid to detect the presence of BPH protein markers to determine whether the subject is afflicted with BPH.

This invention further provides a method for determining whether a subject is afflicted with Benign Prostate Hyperplasia (BPH) comprising obtaining a serum sample from the subject and analyzing the serum sample to detect the presence of BPH protein markers to determine whether the subject is afflicted with BPH.

Finally, this invention provides a diagnostic kit for detecting Benign Prostate Hyperplasia (BPH) markers comprising BPH protein markers and instructions for use and a diagnostic kit for detecting Benign Prostate Hyperplasia (BPH) markers comprising antibodies to PSP61, IwPSA, and α s1-casein and instructions for use.

### BRIEF DESCRIPTION OF THE FIGURES

FIG. 1 is a two dimensional electrophoresis analysis of secretory proteins from human expressed prostate secretion (EPS) in BPH and BPH-free controls. 200 μg of protein was separated according to its isoelectric points (pI, pH 3-10) and molecular weight, and the gel was Silver-stained. One representative gel from young age group (A), age-matched group (B) and BPH patients (C) is shown. Arrows indicate protein spots present in BPH sample only (spots 1-3), highly expressed in BPH (spot 4) and commonly expressed in both BPH and normal control (spots or spot tails 5-8).

FIG. 2 shows spectra of spot 1 (isoform of PSP94, [iPSP94], i.e. PSP61) by ESI-MS/MS. The MS/MS ions are shown at the top of the diagram, while the bottom of the diagram contains the MasEnt43 spectrum.

FIG. 3 is the fragmental sequence of spot 1 (iPSP94, [i.e. PSP61]) by ESI-MS/MS, which was determined to be PSP94 but only 61 amino acids after database searching, thus, PSP61.

FIG. 4 shows the spectra of spot 2 (serum Albumin) by ESI-MS/MS. The MS/MS ions are shown at the top of the diagram, while the bottom of the diagram contains the MasEnt43 spectrum.

FIG. 5 is the fragmental sequence of spot 2 (Serum albumin) by ESI-MS/MS. The MS/MS ions are shown at the top of the diagram, while the bottom of the diagram contains the MasEnt43 spectrum.

FIG. 6 shows the fragmental sequence of low molecular weight PSA (IwPSA).

FIG. 7 is the spectra of αs1-casein in spot 3. The MS/MS ions are shown at the top of the diagram; the bottom of the diagram contains the MasEnt43 spectrum.

FIG. 8 is the fragmental sequence of αs1-casein in spot 3.

FIG. 9 is a spectrum of the peptides of spot 5 by MALDI-MS. The spectrum was made in reflectron mode in the mass range 600 to 3400 Da. The spectrum of the extracted peptides was searched against a database and was identified as Zinc alpha-2 glycoprotein.

FIG. 10 is a spectrum of the peptides of spot 6 by MALDI-MS. The spectrum was acquired in reflectron mode in the mass range 600 to 3400 Da. The spectrum of the extracted peptides was searched against a database was identified as β-microseminoprotein (PSP94).

FIG. 11 is a spectrum of the peptides of spot 8 by MALDI-MS. The spectrum was acquired in reflectron mode in the mass range 600 to 3400 Da. The spectrum of the extracted peptides was searched against a database and was identified as prostatic acid phosphatase (PAP).

FIG. 12 is the spectrum of spot 4 (Lactoferrin) by MALDI-MS. The MS/MS ions are shown at the top of the diagram; the bottom of the diagram contains the MasEnt43 spectrum.

FIG. 13 is the fragmental sequence of spot 4 (Lactoferrin) by ESI MS/MS, which was determined as Lactoferrin after database searching.

FIG. 14 shows immunostaining of α s1-casein in human normal prostate, BPH, and prostate adenocarcinoma specimens (A x100; B x200; C, D, E, F, x400).

FIG. 15 is α s1-casein immunoreactivity in human normal prostate, BPH and prostate cancer. All of the normal prostate and 70% of prostate cancer samples are negative to α s1-casein reactivity. Only 30% of prostate cancer specimens show mild to moderate α s1-casein immunoreactivities, respectively. In contrast, 91% of BPH specimens show moderate to strong α s1-casein immunoreactivities with 36% at ++ and 55% at +++ levels.

### DETAILED DESCRIPTION OF THE INVENTION

Definitions

As used in this application, except as otherwise expressly provided herein, each of the following terms shall have the meaning set forth below.

As used herein, “subject” shall mean any animal, such as a primate, mouse, rat, guinea pig or rabbit. In the preferred embodiment, the subject is a human.

As used herein, “protein markers” shall mean proteins found whose levels correlate with the type cancer. “BPH protein markers” are proteins whose levels correlate with the diagnosis of BPH.

Embodiments of the Invention

This invention provides a method for determining whether a subject is afflicted with Benign Prostate Hyperplasia (BPH) comprising obtaining a sample of prostatic fluid from the subject and analyzing the sample of prostatic fluid to detect the presence of BPH protein markers to determine whether the subject is afflicted with BPH. In the preferred embodiment, the subject is a human male. The sample of prostatic fluid may be obtained by expressing the digital massaging the prostate gland of a patient through rectal approach. In one embodiment, the marker proteins are detected using 2-D gel electrophoresis method and mass spectrometric analyses. Specific antisera against these marker proteins are raised. The protein markers may be detected using antibodies to the protein markers. In the preferred embodiment, the protein markers comprise PSP61, IwPSA or α s1-casein.

This invention further provides a method for determining whether a subject is afflicted with Benign Prostate Hyperplasia (BPH) comprising obtaining a serum sample from the subject; and analyzing the serum sample to detect the presence of BPH protein markers to determine whether the subject is afflicted with BPH. In the preferred embodiment, the subject is a human male and the protein markers comprise PSP61, IwPSA or α s1-casein. In another embodiment, the protein markers may be detected using an immunoradiometric assay using antibodies. These antibodies may be monoclonal antibodies to PSP61, IwPSA or α s1-casein.

This invention further provides a diagnostic kit for detecting Benign Prostate Hyperplasia (BPH) markers. In one embodiment, the diagnostic kit comprises BPH protein markers and instructions for use. In another embodiment, the diagnostic kit for detecting Benign Prostate Hyperplasia (BPH) markers comprises antibodies to PSP61, IwPSA, and α s1-casein and instructions for use. In one embodiment, the antibodies are monoclonal antibodies. In another embodiment, the antibodies are attached to a substrate.

The following experimental details are intended to be exemplary of the practice of the present invention, and should not be construed to limit the scope of the invention in anyway.

Experimental Details

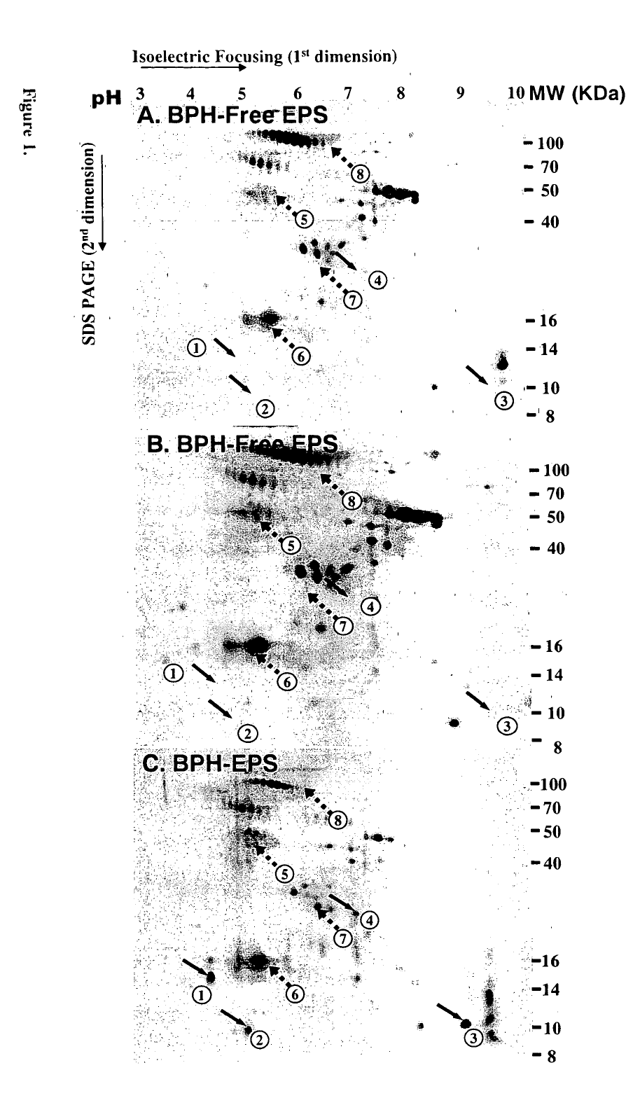

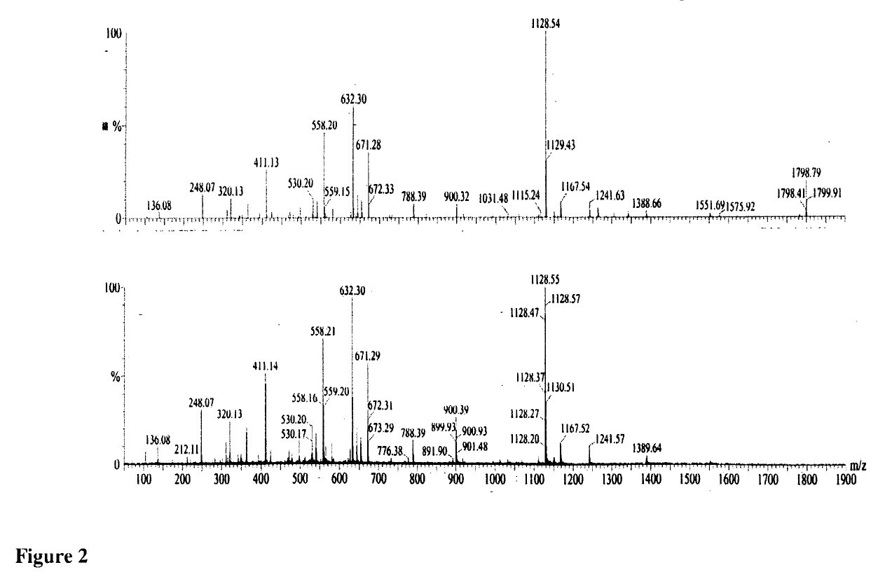

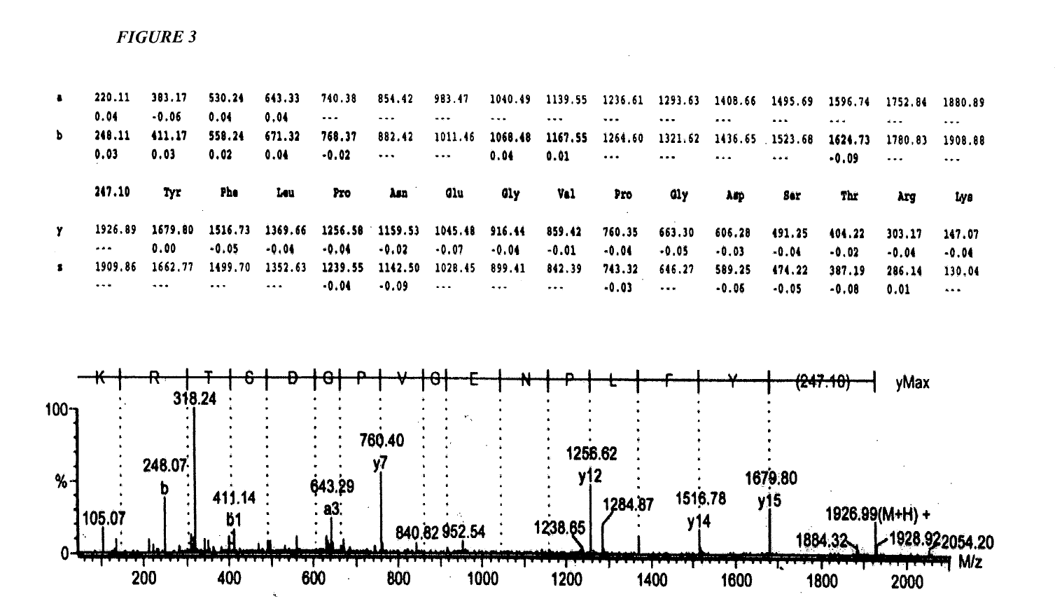

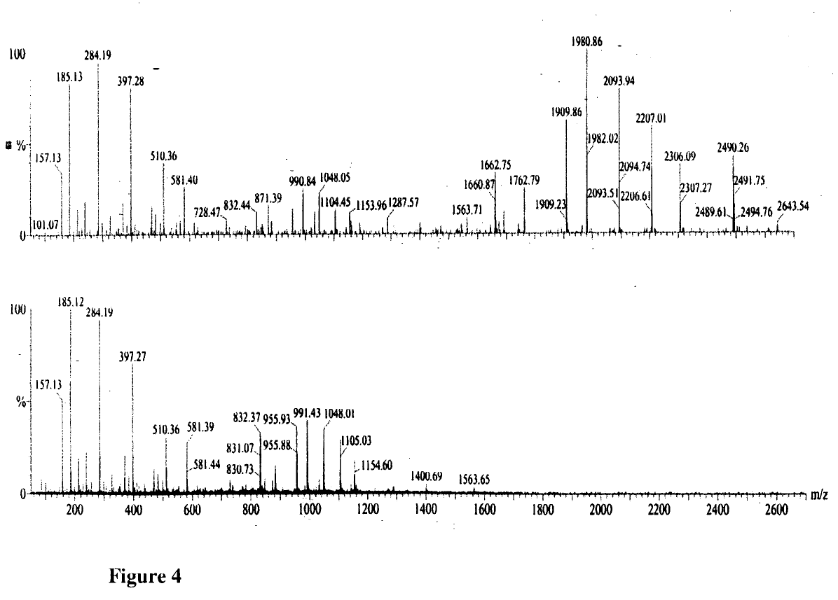

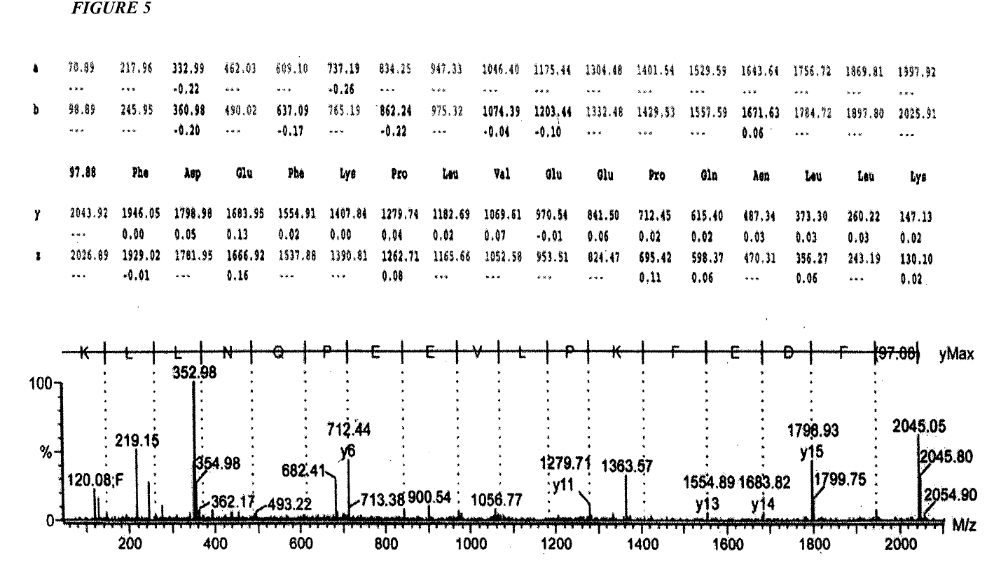

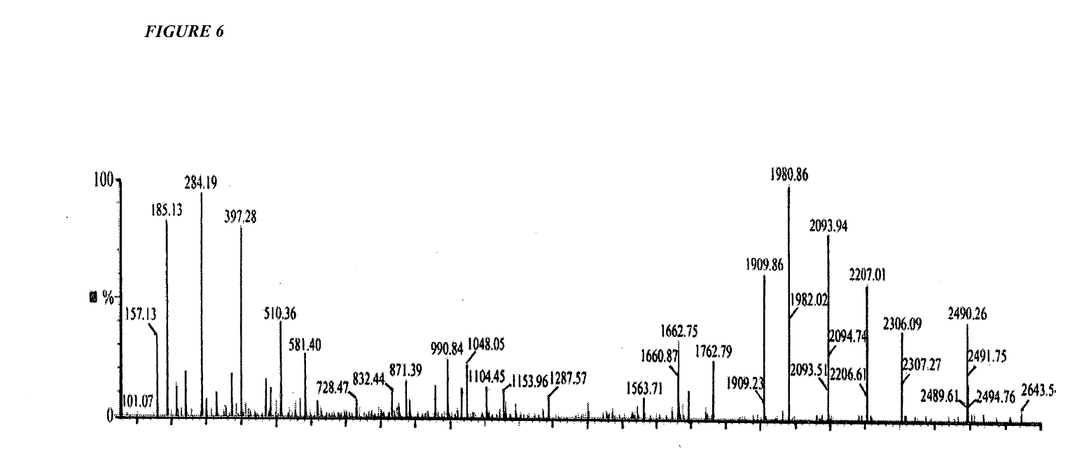

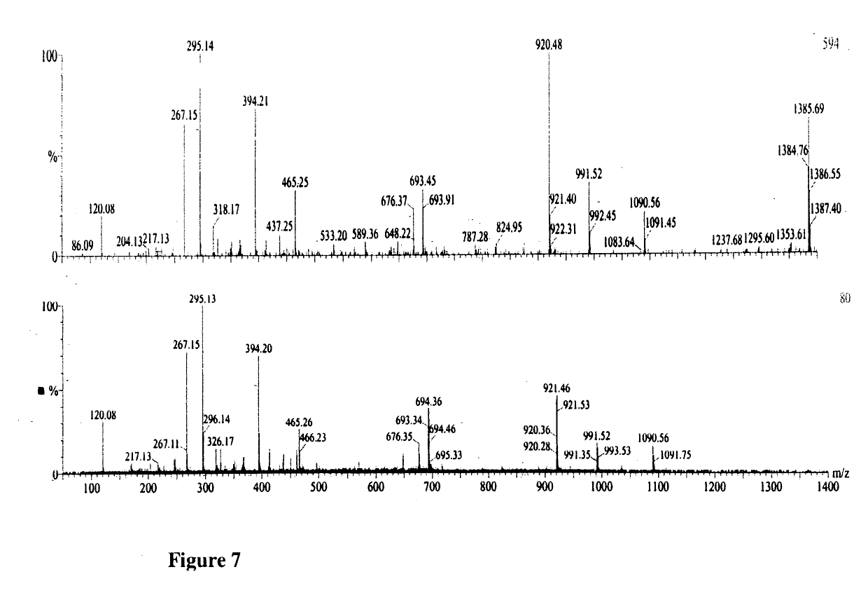

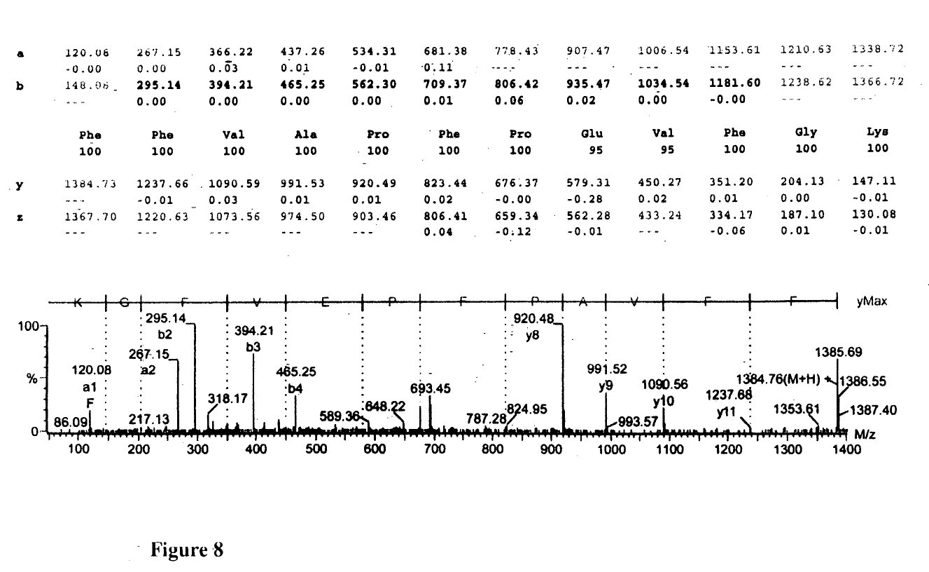

Extensive studies to analyze the prostatic secretions obtained by direct massage of prostate gland through digital rectal examination (DRE) have been performed. The secretions collected from the process are known as expressed prostatic secretions (EPSs). The EPS samples of normal human prostate (young and age-matched controls) and BPH were mixed with protease inhibitors respectively and stored frozen until use. The comparative analysis of EPS of normal prostate and BPH by 2-D gel electrophoresis revealed that a number of proteins were differentially expressed in secretions from BPH. Specifically, three major spots, designated as spots 1, 2 and 3 were found only in BPH samples while totally absent or expressed at very low to undetectable levels from the normal/age-matched samples (FIG. 1). Although there were also additional differentially expressed spots (i.e. spot 4 etc), the current focus is on the three major spots (spots 1-3). Analysis of these differentially expressed spots by mass spectrometric examination and mass sequencing (with the assistance of the Australian Proteome Analysis Facility [APAF] at Macquarie University in Sydney) revealed that the spot 1 appeared to be an “isoform” of PSP94 (iPSP94; MW 14 kDa, pI 4.5) or beta-microseminoprotein, also known as inhibin-like polypeptide or β-inhibin. We have further characterized this protein and found that this protein in fact contains 61 amino acids (instead of 94 amino acids for PSP94) (See FIGS. 2 and 3), thus should be more appropriately called PSP61. The spot 2 (MW of 9 kDa, pI 5-5.5) turned out to be serum albumin (FIGS. 4, 5), while spot 3 (MW 10 kDa, pI 8.5-9.3) contained two proteins; one was low molecular weight PSA (IwPSA) (FIG. 6) while the other was alpha s1-casein (FIGS. 7, 8).

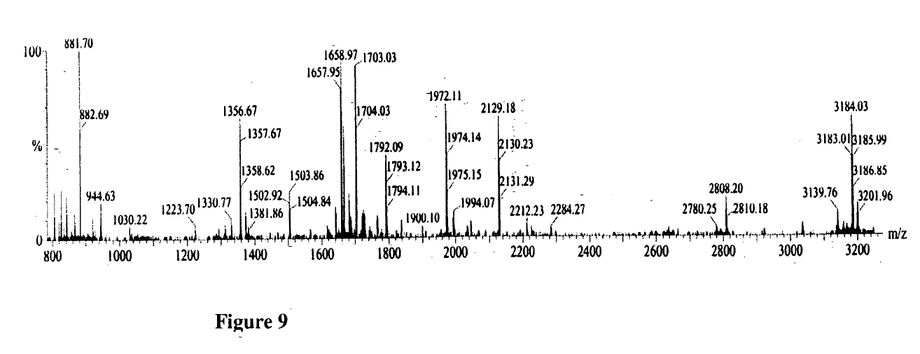

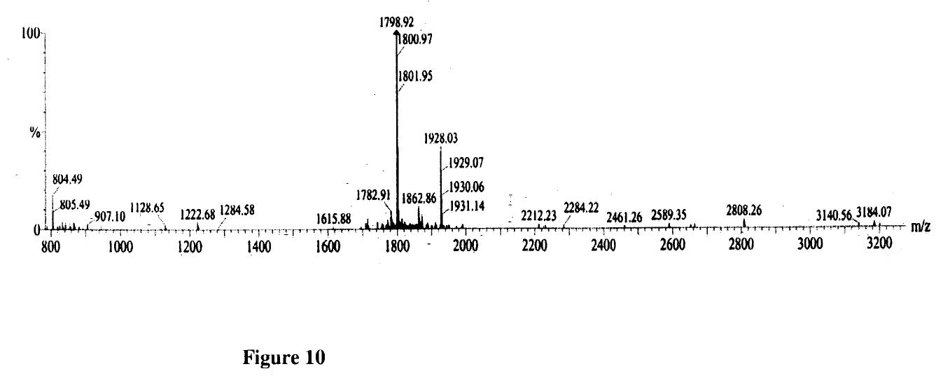

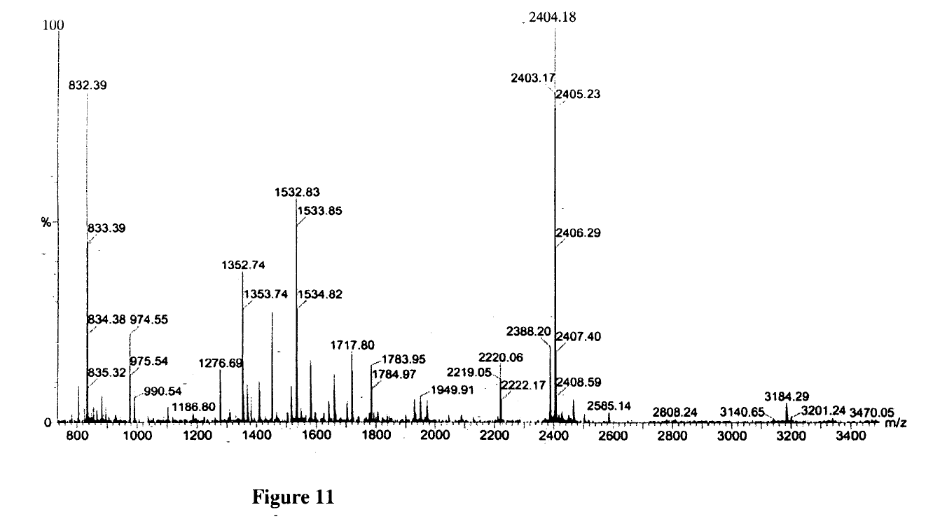

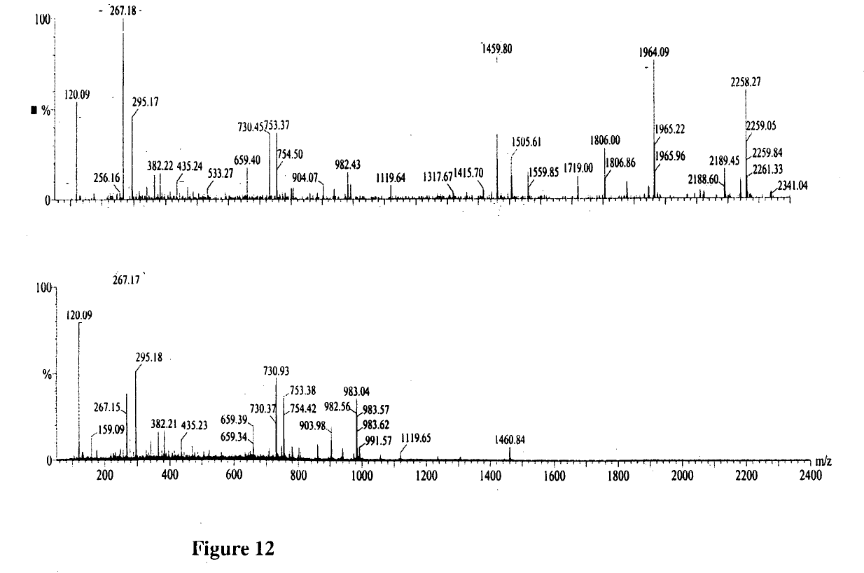

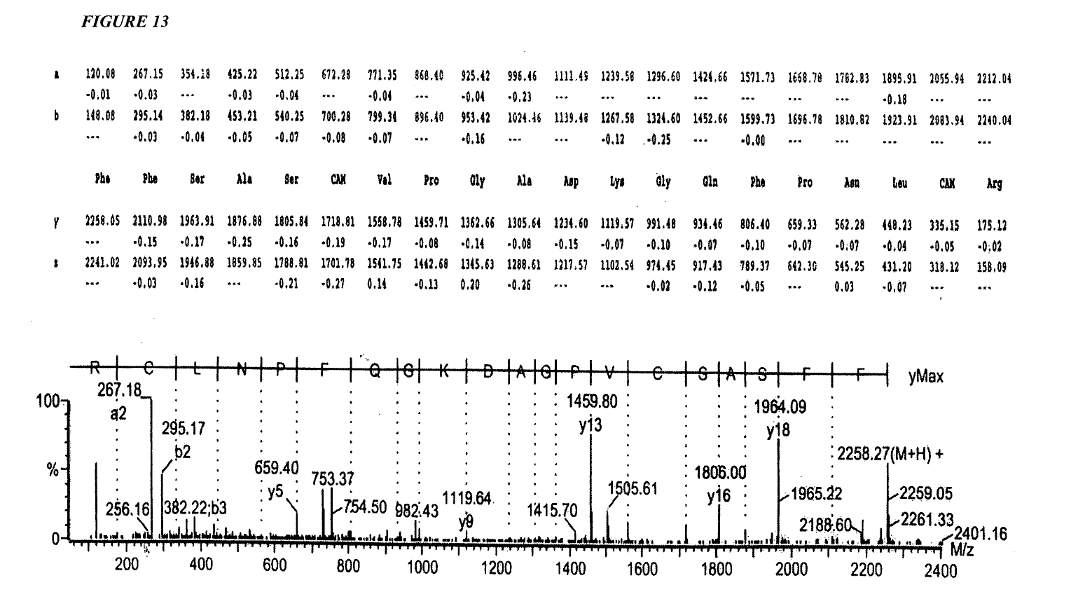

To check the validity and reliability of the assay, several major protein spots which were present in both normal, age-matched controls and BPH samples were analyzed. For example, Spot 5 was Zinc α-2-glycoprotein (FIG. 9), while spots 6 to 8 were PSP94 (FIG. 10), PSA [data not shown, confirmed by Western blotting] and prostatic acid phosphatase [PAP] (FIG. 11) respectively. It was further noted that spot 4 in fact was lactoferrin (FIGS. 12, 13). These proteins had been confirmed with specific antisera and they have been reported as secretory components of the prostate gland.

In the present study, both young adult male (under 40 years) EPS samples, and age-matched BPH-free EPS were used. EPS's from patients with confirmed prostate cancer were not included, the rationale being that most patients with prostate cancer would invariably have varying degrees of BPH as well. Thus, inclusion of EPS's from prostate cancer patients would only serve to confuse the signal of BPH unnecessarily at this early stage. The supernatant of three prostate cancer cell lines, LNCaP (PSA+), PC3 (PSA−) and DU145 (PSA−), were analyzed by 2-D gel and found that LNCaP (as well as other two lines) were negative to IwPSA, while either negative or only a trace amount of PSP94 was detected in all three lines. In humans, it has been reported that PSP94 is down-regulated in prostate cancer. This would serve to support our hypothesis that IwPSA and PSP94 and PSP61 are not expressed in any significant way by prostate cancer cells.

I. Identification and Characterization of Specific Marker Proteins in Secretion of BPH.

A. Collection of Prostatic Secretions

Expressed prostatic secretions (EPSs) were collected from 10 men aged 40 or less (normal), 50 age-matched controls and from 50 men aged between 60-70 years old with confirmed diagnosis of BPH. Before commencing on any treatment protocols, the EPS samples (each about 1 ml) from both the normal, age-matched controls (BPH free) and BPH patients (with informed consent) was obtained and mixed with protease inhibitors respectively and stored frozen (−80° C.) until use. They were designated as BPH-free (normal and age-matched controls) and BPH-EPS (BPH) respectively.

B. Two-Dimensional Gel Electrophoresis

Two-dimensional gel electrophoresis of EPS from normal, age-matched controls and BPH patients were performed using the protocols (31) adapted from the book edited by Wilkins et al., as summarized in the following passages using the IPGphor Isoelectric Focusing System and vertical SDS-PAGE.

### 1. First-Dimension Isoelectric Focusing (IEF)

Isoelectric focusing was carried out using Immobilized pH Gradient (IPG) Strip Gel (Amersham Pharmacia Biotech). Five μl of EPS was dissolved or diluted in 2501 μl of rehydration solution (8M urea, 2% CHAPS, 0.5% IPG Buffer), which was added to IPG strip and allowed to rehydrate for 14 h. The EPS samples were run at an increasing voltage from 100V (2 h), 500V (1 h), 1000V (1 h) to 8000V (4 h). In this first dimensional IEF, the proteins were separated according to their pH.

### 2. Second-Dimension SDS-PAGE

The IPG strip was equilibrated for 30 minutes in equilibration buffer (50 mM Tris-Cl, 6M urea, 30% glycerol, 2% SDS, DTT 10 mg/ml or iodoacetamide 25 mg/ml and tracking dye). Subsequently, the IPG strip was placed to vertical SDS-PAGE system (Hoefer SE 600) and the proteins separated by electrophoresis according to their molecular weights. The resulting gels were stained with silver using a silver-staining kit (Amersham). Comparison of 2-D gels of EPS from young and age-matched controls and BPH with the help of ImageMaster 2D Elite gel analysis software (Amersham) to reveal differentially expressed protein profiles.

C. Mass Spectrometric Analysis

The protocols as outlined in Young C Y F, Seay T, Higen K, Charlesworth M C, Roche P C, Klee G G, Tindall D, Prostate [Suppl] 7:17-24 (1996) were followed.

### 1. Matrix-Assisted Laser Desorption-Ionization Mass Spectrometric (MALDI-MS) Method

For Mass Spectrometric Analysis, electrophoretograms can either be stained by silver or Coomassie blue. The differentially expressed protein spots on 2-D gels were identified and cut out for mass spectrometric analysis. For mass spectrometric analysis we solicited the assistance of Australian Proteome Analysis Facility (APAF). The MALDI-MS method was employed. The isolated protein spots first underwent an in-gel tryptic digestion at 37C. The resulting peptides were extracted from the gel with a 10% acetonitride, 1% TFA solution and subjected to a ZipTip clean-up. The peptides were eluted from the matrix and spotted onto a MALDI target and allowed to air dry. MALDI-MS was then performed with a Micromass ToF Spec 2E Times-of-Flight Mass Spectrometer. A nitrogen laser (337 nm) was used to irradiate the samples and the spectra were acquired through reflectron mode in the mass ranging from 600 to 3500 Da. A near point calibration was applied and this gave a typical mass accuracy of ˜100 ppm or less. The monoisotopic peak list of the peptides from the sample will then be achieved (see FIGS. 10-12). The peptide mass fingerprinting was searched against Homo sapiens using SWISS-PROT and TREMBL with Peptident. This provided the most likely candidate proteins based on the peptide fingerprinting.

### 2. Electrospray Ionization Mass Spectrometry/Mass Sequencing (ESI-MS/MS)

The nature of the differentially expressed protein spots was further confirmed by ESI-MS/MS. After the 16 h tryptic digestion, the resulting peptides were purified using a ZipTip to concentrate and to desalt. The samples were analyzed by ESI-TOF MS/MS using a Micromass Q-TOF MS equipped with a nanospray source and data manually acquired using borosilicate capillaries. Data were acquired over the m/z range 400-1800 to select peptides for MS/MS analysis. After peptides were selected the machine was switched to mass spectra/mass sequence mode and data collected over the m/z range 50-2000 with variable collision energy settings. Spectra of peptide fragmentation and fragment ions (y-type ions were used for sequencing of peptide concerned) were generated. From these data collected, sequence of amino acid of the peptide concerned was determined. Based on the data of amino acid sequence of selected peptide, the most likely proteins were determined.

II. Cellular Sources of Specific Marker Proteins

Having identified the nature of these differentially expressed protein by 2-D gel and mass spectrometry, the cellular sources of the three specific proteins were examined by immunohistochemical and in situ hybridization methods and evaluated the specificity of these protein markers for BPH.

A. Generation of Anti-Sera

In order to establish the cellular sources of these proteins, anti-sera against these proteins were generated. For this part, the services of the Alpha Diagnostic International, San Antonio, Tex., USA were used to generate antibodies specific to these marker proteins based on their known amino acid sequences as determined by mass sequencing.

B. Immunohistochemistry (IHC)

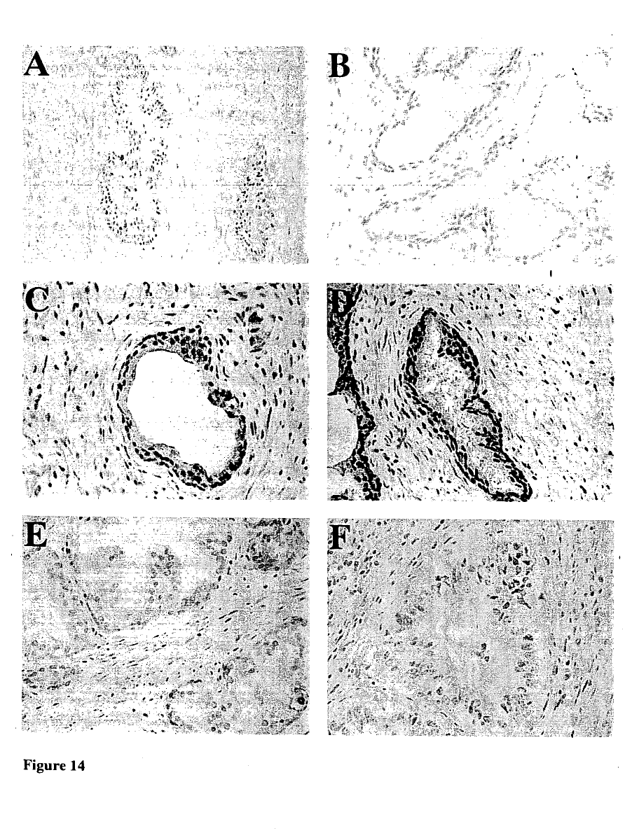

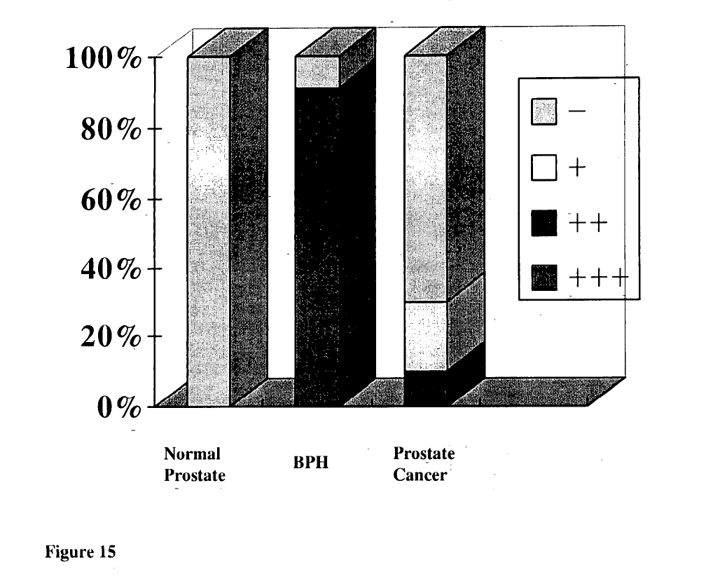

The specificity of anti-sera was first checked against the corresponding differentially expressed protein spots. These anti-sera were then used for IHC on normal human prostate, age-matched controls, BPH as well as prostate cancer samples. The results showed that prostatic epithelial cells of BPH showed positive reactivity to these marker proteins while the stromal tissue was negative (FIGS. 14, 15). This demonstrated that the specific marker proteins were secreted by BPH epithelial cells.

III. Detection of Protein Markers

The final stage is to examine whether PSP61, IwPSA and alpha s1-casein are present in serum in BPH patients and to develop a methodology to detect the presence of these proteins in serum for diagnosis of BPH and to assess the correlation of the newly found markers with PSA levels especially in marginal cases.

A. Introduction

Since blood test is one of the most convenient means of clinical examination, the final step of this project is to examine if these three proteins can be detected in the blood of BPH patients and whether they are specific for BPH patients. PSA levels will also be tested in parallel and the association between PSA levels and the levels of these three proteins will be investigated. We anticipate the results to be positive for PSA and PSP94 as they are known to be present in serum. It is expected that the variants of these molecules (i.e. PSP61, IwPSA and α-s1-casein) will also make their way to the blood and thus detectable in the serum. When confirmed, this will prove to be extremely useful for diagnosis of early BPH before the emergence of clinical symptoms. Production of testing kits may then be considered.

B. Collection of Blood For Assay

Venous blood (20 ml) from 100 patients with newly diagnosed BPH is collected in a standard manner. For comparison purposes, blood of 20 normal men (aged under 40 years) and 50 age-matched normal controls is also collected. Further, blood samples from 50 patients with PSA levels ranging from 4-10 nm/ml are collected for comparison purposes. Samples are left at room temperature for one hour and then centrifuged at 3500 rpm for 10 minutes to separate serum from the other blood components. Samples are then be stored at 20° C. until use.

C. Determination of PSP61, IwPSA and αs1-casein Serum Concentration

The samples are analyzed by immunoradiometric assay (Tandem-R PSA, Hybritech, Inc.) or methods as depicted by Labrie et al. (34), for regular serum free PSA and total PSA assay. These methods are adapted for assaying serum IwPSA, PSP61 and αs1-casein by replacing its original antibodies with specific monoclonal antibodies generated (i.e. IwPSA, PSP61 and αs1-casein). The data obtained are compared with data of regular PSA or PSP94. Once the assaying methodology has been established, a larger scale serum screening is carried out to determine the specificity of these proteins as markers for BPH.

D. Correlation of Levels of Specific BPH Markers and Serum PSA Levels with Special Reference to Marginal Elevation of PSA Levels (i.e. 4-10 ng/ml)

The PSA levels of all patients (100) with confirmed BPH are assessed and compared their PSA levels with specific BPH markers as established in this study. The BPH specific markers in patients with marginal elevation of PSA levels are also assessed by determining their serum levels of specific BPH markers (i.e. PSP61, IwPSA and αs1-casein). By comparing the PSA levels with specific BPH markers in BPH patients and also those from patients with marginal elevation of serum PSA, it is determined whether the latter group has prostate cancer. Patients with concurrent elevation of PSA (marginal elevation) and specific markers are likely to have BPH only. On the other hand, patients with decreased or undetectable levels of specific BPH markers would more likely to have prostate cancer.

The following references have been referred to throughout the specification. The entire contents of these references is hereby incorporated by reference herein as background and illustrative of the state of the art of the invention.

### REFERENCES

1. Berry S J, Coffey D S, Walsh P C, Ewing L L, J. Urol. 132:474-479 (1984).
2. Carter H B and Coffey D S, The Prostate 16:39-48 (1990).
3. McNeal J E, Invest. Urol. 15:340-345 (1978).
4. McNeal, J E, “The prostate gland: Morphology and pathobiology,” Monogr. in Urol. vol 4. No 1 (1983).
5. Djavan B, Nadersbacher S, Klingler H C, Ghawidel K, et al., Tech. Urol. 5:12-20 (1999).
6. Holtgrewe H L., “Transurethral prostatectomy,” Urol. Clin. North Am. 22:357-368 (1995).
7. Arai Y, Aoki Y, Okubo K, Maeda H, Terada N, et al., J. Urol. 164:1206-11 (2000).
8. Dixon C M and Lepor H, Benign Prostatic Hyperplasia, Z. Petrovich and L. Baert (eds.). Springer-Verlag, 193-204 (1993).
9. Gromley G J, Stoner E, Bruskewitz R C et al., N. Eng. J. Med. 327:1185-1191 (1992).
10. Vermeulen A, Giagulli V A, Scheppe P, Buntinx A, Stoner E, The Prostate 14:45-53 (1989).
11. Geller J, J. Clin. Endocr. Met. 71:1552-1555 (1990).
12. Kirby R and Christmas T, Benign Prostatic Hyperplasia. Gower Med. Publ. (1993).
13. Dupont A, Cusan L, Gomez J L, Thibeault M M, Tremblay M, Labrie F., J. Urol., 146:1064-7 (1991).
14. Partin A W, Oesterling J E, J. Urol. 152:1358-1368 (1994).
15. Lee C S, Oesterling J E (1995). Sem. Surg. Oncol., 11:23-35.
16. Barichello M, Gion M, Bonazza A, et al., Eur. Urol., 27:295-300 (1995).
17. Mikolajczyk S D, Millar L S, Wang T J, Rittenhouse H G, Wolfert R L, Marks L S, Song W T, Wheeler T M and Slawin K M, Urol., 55:41-45 (2000).
18. Wang T J, Slawin K M, Rittenhouse H G, Millar L S and Mikolajczyk S D, Eur. J. Biochem., 267:4040-4045 (2000).
19. Mikolajczyk S D, Millar L S, Marker K M, Wang T J, Rittenhouse H G, Marks L S, Slawin K M, Prostate, 45:271-276 (2000).
20. Wang Y Z and Wong Y C, “Sex Hormone-Induced Prostatic Carcinogenesis In The Noble Rat: The Role Of Insulin-Like Growth Factor-1 (IGF-1) and Vascular Endothelial Growth Factor (VEGF) in the Development Of Prostate Cancer,” Prostate, 35:165-177 (1998).
21. Xie W, Wong Y C, Tsao S W and Wong N S, “Expression Of A Kallikrein-Like Protein In Prostatic Intraepithelial Neoplasia In Ventral Prostate Of The Noble Rat,” Prostate, 42:8-17 (2000).
22. Wong Y C, Wang Y Z and Tam N NC, “The Prostate Gland And Prostate Carcinogenesis,” Italian J. Anat. Embryol., 103 (Suppl. 1) n. 4:237-252 (1998).
23. Tam N N C, Chung S S M, Lee D T W and Wong, Y C, “Aberrant Expression Of Hepatocyte Growth Factor And Its Receptor, c-Met, During Sex Hormone-induced Prostatic Carcinogenesis In The Noble Rat,” Carcinogenesis, 21:2183-2191 (2000).
24. Wong Y C, Xie W and Tsao S W, “Structural Changes And Alteration In Expression Of TGF-β1 And Its Receptors In Prostatic Intraepithelial Neoplasm (PIN) In The Ventral Prostate Of Noble Rats,” Prostate, 45:289-298 (2000).
25. Wong Y C, Tam N NC, “Dedifferentiation Of Stromal Smooth Muscle As A Factor In Prostate Carcinogenesis,” Differentiation, 70:633-45 (2002).
26. Ouyang X S, Wang X H, Lee D T W, Tsao S W and Wong Y C, “Up-regulation of TRMP-2, MMP-7 and Id-1 During Sex Hormone-induced Prostate Carcinogenesis In The Noble Rat,” Carcinogenesis, 22:965-973 (2001).
27. Ouyang, X S, Wang X H, Lee T W, Tsao S W and Wong Y C, “Overexpression of Id-1 in Prostate Cancer,” J. Urology, 167: 2598-2602 (2002).
28. Ouyang X S, Wang X H, Ling M T, Wong H L, Tsao S W, and Wong Y C, “Id-1 Stimulates Serum Independent Prostate Cancer Cell Proliferation Through Inactivation of p16^{INK4a}/pRB Pathway,” Carcinogenesis, 23:721-725 (2002).
29. Ling M T, Wang X H, Ouyang X S, Lee T K W, Fan T Y, Xu K X, Tsao S W and Wong Y C, “Activation of MAPK Signaling Pathway Is Essential for Id-1 Induced Serum Independent Prostate Cancer Cell Growth,” Oncogene, 21:8498-8505 (2002).
30. Ling M T, Wang X H, Ouyang X S, Xu K X, Tsao S W and Wong Y C, “Id-1 Expression Promotes Cell Survival Through Activation of NF-KB Signaling Pathway In Prostate Cancer Cells,” Oncogene (In Press) (2003).
31. Young C Y F, Seay T, Higen K, Charlesworth M C, Roche P C, Klee G G, Tindall D, Prostate [Suppl] 7:17-24 (1996).
32. K X Xu, X Wang, M T Ling, D T W. Lee, T Y Fan, F L Chan, J J W Xuan, S W Tsao, Y C Wong, “Identification of a Specifically Expressed Modified Form Of Novel PSP-94 Protein in the Secretion of Benign Prostatic Hyperplasia,” Electrophoresis, 24:1311-1316 (2003).
33. Chan P S F, Chan L W, Xuan J W, Chin J L, Choi H L and Chan F L, Prostate, 41:99-109 (1999).
34. Wilkins M R, Williams K L, Appel R D and Hochstrasser D F (eds.) Proteome Research: New Frontiers in Functional Genomics, Principles and Practice, Springer (1997).
35. Labrie F, Dupont A, Suburu R, Cusan L, Tremblay M, Gomez J L and Emond J., J. Urol., 147:846-852 (1992).

1. A method for determining whether a subject is afflicted with Benign Prostate Hyperplasia (BPH) comprising:
(a) obtaining a sample of prostatic fluid from the subject; and
(b) analyzing the sample of prostatic fluid to detect the presence of BPH protein markers to determine whether the subject is afflicted with BPH.

2. The method in accordance with claim 1, wherein the subject is a human male, and the sample is obtained by expressing the prostate gland of the subject.

3. The method in accordance with claim 1, wherein the sample of prostatic fluid is analyzed by 2-D gel electrophoresis and mass spectrometry.

4. The method in accordance with claim 1, wherein the BPH protein markers are detected using antibodies to the BPH protein markers.

5. The method in accordance with claim 1, wherein the protein markers comprise PSP61, IwPSA or α s1-casein.

6. A method for determining whether a subject is afflicted with Benign Prostate Hyperplasia (BPH) comprising:
(a) obtaining a serum sample from the subject; and
(b) analyzing the serum sample to detect the presence of BPH protein markers to determine whether the subject is afflicted with BPH.

7. The method in accordance with claim 6, wherein the subject is a human male.

8. The method in accordance with claim 6, wherein the protein markers are detected using an immunoradiometric assay.

9. The method in accordance to claim 6, wherein the BPH protein markers are detected using antibodies to the BPH protein markers.

10. The method in accordance with claim 6, wherein the protein markers comprise PSP61, IwPSA or α s1-casein.

11. A diagnostic kit for detecting Benign Prostate Hyperplasia (BPH) markers comprising BPH protein markers and instructions for use.

12. A diagnostic kit for detecting Benign Prostate Hyperplasia (BPH) markers comprising antibodies to PSP61, IwPSA, and α s1-casein and instructions for use.

13. The diagnostic kit in accordance with claim 12, wherein the antibodies are monoclonal antibodies.

14. The diagnostic kit in accordance with claim 12, wherein the antibodies are attached to a substrate.
